(19) United States
(12) Patent Application Publication
(10) Pub. No.: US 2026/0351404 A1
(43) Pub. Date: Nov. 5, 2026

(54) ATTENTIONAL GENERATIVE MULTIMODAL NETWORK FOR PAIN ESTIMATION

(71) Applicant: UNIVERSITY OF SOUTH FLORIDA, Tampa, FL (US)

(72) Inventors: Md Sirajus SALEKIN, Tampa, FL (US); Ghadh ALZAMZMI, Tampa, FL (US); Dmitry GOLDGOF, Tampa, FL (US); Yu SUN, Tampa, FL (US); Thao HO, Tampa, FL (US); Peter Randolph MOUTON, Tampa, FL (US)

(21) Appl. No.: 19/105,017

(22) PCT Filed: Aug. 21, 2023
(86) PCT No.: PCT/US2023/072593

§ 371 Date: Feb. 19, 2025

Related U.S. Application Data
(60) Provisional application No. 63/399,563, filed on Aug. 19, 2022.

(51) Int. Cl. G16H 50/20; G06N 7/01

(52) U.S. Cl. CPC G16H 50/20; G06N 7/01

(57) ABSTRACT

Methods and systems for pain assessment are disclosed. The methods and systems include: obtaining a trained first, second, and third artificial intelligence (AI) models; obtaining sensor data for each modality of multiple modalities for a sequence length; determining a latent feature space in the sequence length for each modality of the multiple modalities based on the first AI model and the sensor data for each modality; generating a common latent space based on the second AI model and the latent feature space of each modality of the multiple modalities; generating a reconstructed latent space for each modality of the multiple modalities based on the common latent space and the second AI model; and determining a pain indication and/or a level of intensity based on the third AI model. Other aspects, embodiments, and features are also claimed and described.

### CROSS-REFERENCE TO RELATED APPLICATION(S)

This application claims the benefit of U.S. Provisional Patent Application Ser. No. 63/399,563, filed Aug. 19, 2022, the disclosure of which is hereby incorporated by reference in its entirety, including all figures, tables, and drawings.

### STATEMENT REGARDING FEDERALLY SPONSORED RESEARCH

This invention was made with government support under R21NR018756 awarded by the National Institutes of Health. The government has certain rights in the invention.

### SUMMARY

The following presents a simplified summary of one or more aspects of the present disclosure, to provide a basic understanding of such aspects. This summary is not an extensive overview of all contemplated features of the disclosure and is intended neither to identify key or critical elements of all aspects of the disclosure nor to delineate the scope of any or all aspects of the disclosure. Its sole purpose is to present some concepts of one or more aspects of the disclosure in a simplified form as a prelude to the more detailed description that is presented later.

In some aspects of the present disclosure, methods, systems, and apparatus for assessing neonatal postoperative pain are disclosed. These methods, systems, and apparatus may include steps or components for: obtaining a trained first artificial intelligence (AI) model, a trained second AI model, and a trained third AI model; obtaining sensor data for each modality of multiple modalities for a sequence length; determining a latent feature space in the sequence length for each modality of the multiple modalities based on the first AI model and the sensor data for each modality; generating a common latent space based on the second AI model and the latent feature space of each modality of the multiple modalities; generating a reconstructed latent space for each modality of the multiple modalities based on the common latent space and the second AI model; and determining a pain indication and/or a level of intensity based on the multiple latent feature spaces, the common latent space, and the reconstructed latent spaces for modalities, and the third AI model.

These and other aspects of the disclosure will become more fully understood upon a review of the drawings and the detailed description, which follows. Other aspects, features, and embodiments of the present disclosure will become apparent to those skilled in the art, upon reviewing the following description of specific, example embodiments of the present disclosure in conjunction with the accompanying figures. While features of the present disclosure may be discussed relative to certain embodiments and figures below, all embodiments of the present disclosure can include one or more of the advantageous features discussed herein. In other words, while one or more embodiments may be discussed as having certain advantageous features, one or more of such features may also be used in accordance with the various embodiments of the disclosure discussed herein. Similarly, while example embodiments may be discussed below as devices, systems, or methods embodiments it should be understood that such example embodiments can be implemented in various devices, systems, and methods.

### BRIEF DESCRIPTION OF THE DRAWINGS

FIG. 1 is a block diagram conceptually illustrating a system for pain assessment according to some embodiments.

FIG. 2A is a flow diagram illustrating an example process for pain assessment according to some embodiments, and FIG. 2B is a flow diagram illustrating another example process for pain assessment according to some embodiments.

FIG. 3 is a flow diagram illustrating an example process for pain assessment system training according to some embodiments.

FIG. 4 is an example conceptual framework for pain assessment according to some embodiments.

FIG. 5 shows t-distributed stochastic neighbor embedding (t-SNE) projection of spatio-temporal features using perplexity of 40.

FIG. 6 is an example influence function-based method integrated into a pain classification model according to some embodiments.

### DETAILED DESCRIPTION

The detailed description set forth below in connection with the appended drawings is intended as a description of various configurations and is not intended to represent the only configurations in which the subject matter described herein may be practiced. The detailed description includes specific details to provide a thorough understanding of various embodiments of the present disclosure. However, it will be apparent to those skilled in the art that the various features, concepts and embodiments described herein may be implemented and practiced without these specific details. In some instances, well-known structures and components are shown in block diagram form to avoid obscuring such concepts.

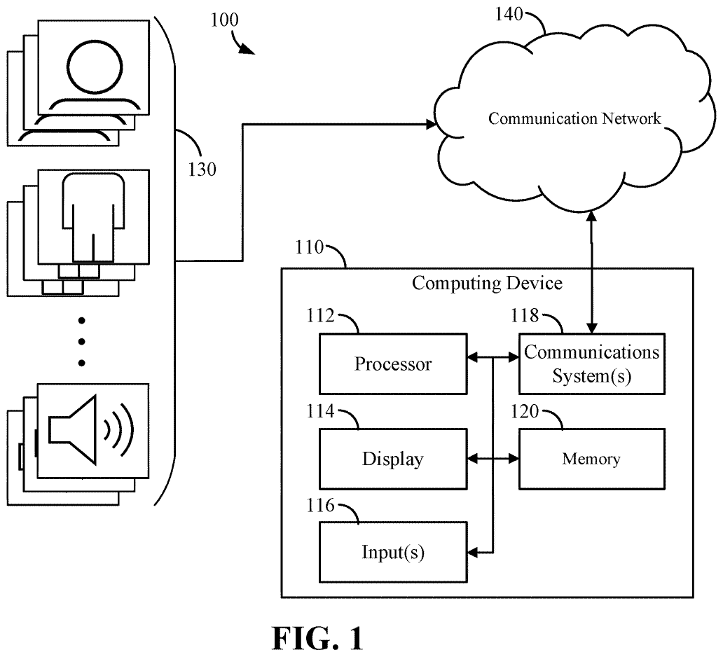

FIG. 1 shows an example 100 of a system for pain assessment in accordance with some embodiments of the disclosed subject matter. As shown in FIG. 1, a computing device 110 can receive runtime sensor data (e.g., images, audio signals, etc.) 130 for each modality during a sequence length for pain assessment using first, second, and third AI models. In further examples, the computing device 110 can receive training sensor data 130 for each modality to train the first AI model and/or the second AI model. In non-limiting scenarios, facial images of the sensor data 130 in the sequence length can be one modality, body images of the sensor data 130 in the sequence length can be another modality, and audio data of the sensor data 130 in the sequence length can be another modality.

In further examples, the computing device 110 can receive the runtime/training sensor data 130 over a communication network 140. In some examples, the communication network 140 can be any suitable communication network or combination of communication networks. For example, the communication network 140 can include a Wi-Fi network (which can include one or more wireless routers, one or more switches, etc.), a peer-to-peer network (e.g., a Bluetooth network), a cellular network (e.g., a 3G network, a 4G network, a 5G network, etc., complying with any suitable standard, such as CDMA, GSM, LTE, LTE Advanced, NR, etc.), a wired network, etc. In some embodiments, communication network 140 can be a local area network, a wide area network, a public network (e.g., the Internet), a private or semi-private network (e.g., a corporate or university intranet), any other suitable type of network, or any suitable combination of networks. Communications links shown in FIG. 1 can each be any suitable communications link or combination of communications links, such as wired links, fiber optic links, Wi-Fi links, Bluetooth links, cellular links, etc.

In further examples, the computing device 110 can be any suitable computing device or combination of devices, such as a desktop computer, a laptop computer, a smartphone, a tablet computer, a wearable computer, a server computer, a computing device integrated into a vehicle (e.g., an autonomous vehicle), a camera, a robot, a virtual machine being executed by a physical computing device, etc. In some examples, the computing device 110 can train and run the first AI model, the second AI model, and/or third AI mode. In other examples, the computing device 110 can train training the first AI model, the second AI model, third AI mode and/or sub-AI models. In the examples, another computing device can run the first AI model, the second AI model, and/or third AI model. In further examples, the computing device 110 can include a first computing device for the first AI model, a second computing device for the second AI model, and a third computing device for the third AI model. It should be appreciated that the training phase and the runtime phase of any combination of the first AI model, the second AI model, and the third AI model can be separately or jointly processed in the computing device 110 (including physically separated one or more computing devices). Although the system described here references three AI models (first, second, and third), alternative realizations of the system could be in the form of a sequence of one or more AI models or a hierarchy of AI models for pain assessment.

In further examples, the computing device 110 can include a processor 112, a display 114, one or more inputs 116, one or more communication systems 118, and/or memory 120. In some embodiments, the processor 112 can be any suitable hardware processor or combination of processors, such as a central processing unit (CPU), a graphics processing unit (GPU), an application specific integrated circuit (ASIC), a field-programmable gate array (FPGA), a digital signal processor (DSP), a microcontroller (MCU), etc. In some embodiments, the display 114 can include any suitable display devices, such as a computer monitor, a touchscreen, a television, an infotainment screen, etc. In some embodiments, the input(s) 116 can include any suitable input devices and/or sensors that can be used to receive user input, such as a keyboard, a mouse, a touchscreen, a microphone, etc.

In further examples, the communications system(s) 118 can include any suitable hardware, firmware, and/or software for communicating information over communication network 140 and/or any other suitable communication networks. For example, the communications system(s) 118 can include one or more transceivers, one or more communication chips and/or chip sets, etc. In a more particular example, the communications system(s) 118 can include hardware, firmware and/or software that can be used to establish a Wi-Fi connection, a Bluetooth connection, a cellular connection, an Ethernet connection, etc.

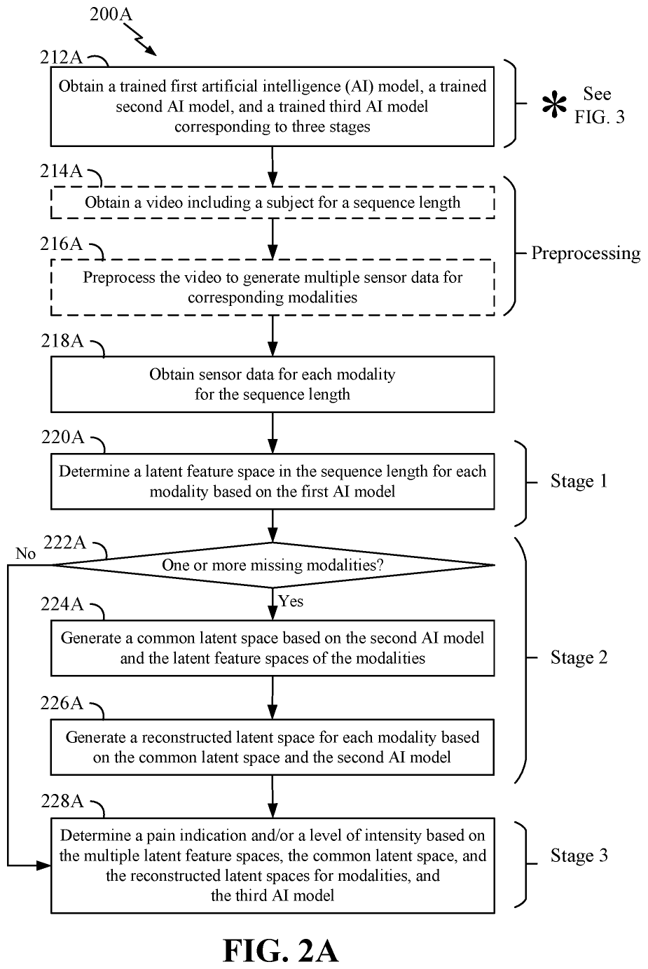

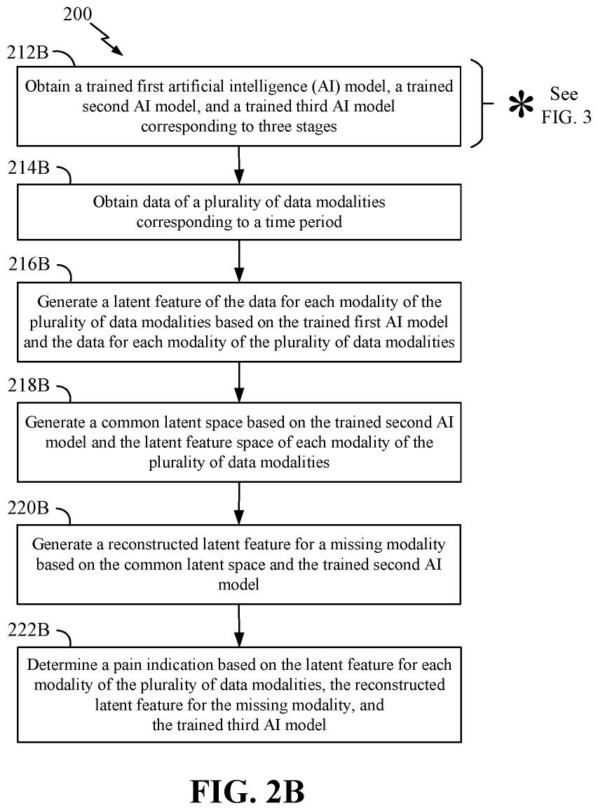

In further examples, the memory 120 can include any suitable storage device or devices that can be used to store image data, instructions, values, AI models, etc., that can be used, for example, by the processor 112 to perform pain assessment/prediction task to present content using display 114, to receive image sources via communications system(s) 118, etc. The memory 120 can include any suitable volatile memory, non-volatile memory, storage, or any suitable combination thereof. For example, memory 310 can include random access memory (RAM), read-only memory (ROM), electronically-erasable programmable read-only memory (EEPROM), one or more flash drives, one or more hard disks, one or more solid state drives, one or more optical drives, etc. In some embodiments, the memory 120 can have encoded thereon a computer program for controlling operation of computing device 110. For example, in such embodiments, the processor 112 can execute at least a portion of the computer program to perform one or more data processing and identification tasks described herein and/or to train/run AI models based on sensory data 130 described herein, present content to the display 114, transmit/receive information via the communications system(s) 118, etc. As another example, processor 112 can execute at least a portion of processes 200A, 200B, and/or 300 described below in connection with FIGS. 2 and/or 3.

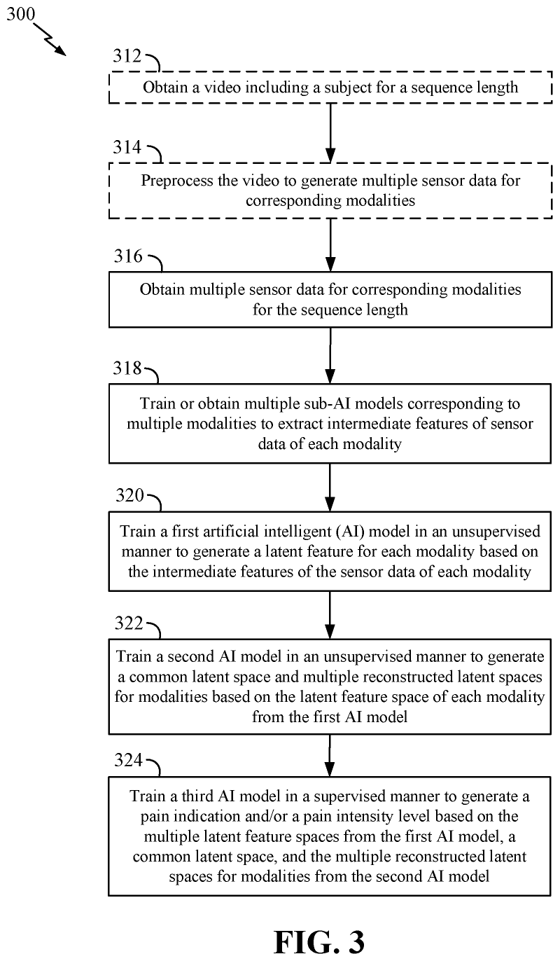

FIG. 2A is a flow diagram illustrating an example process 200A for pain assessment in accordance with some aspects of the present disclosure. As described below, a particular implementation can omit some or all illustrated features/steps, may be implemented in some embodiments in a different order, and may not require some illustrated features to implement all embodiments. In some examples, an apparatus (e.g., computing device 110) in connection with FIG. 1 can be used to perform the example process 200A. However, it should be appreciated that any suitable apparatus or means for carrying out the operations or features described below may perform the process 200A. The process 200A is generally directed to a runtime stage using one or more trained artificial intelligence (AI) models. Training the AI models is described in connection with FIG. 3. In a non-limiting scenario, the process 200A can be used for postoperative pain assessment and/or intensity. However, the process 200A can be used for any other suitable purposes (e.g., preoperative pain assessment, pain prediction, etc.).

At step 212A, the process can obtain a trained first AI model, a trained second AI model, and a trained third AI model corresponding to three stages (e.g., stage 1: spatio-temporal feature detection, stage 2: joint feature distribution detection, and stage 3: attentional feature fusion). In some examples, step 212A can be performed on a different apparatus (e.g., a different processing resource) than the apparatus used to perform other steps in FIG. 2. In another example, step 212A as well as other steps in FIG. 2 can be performed on the same apparatus. The training of the AI models is further described in connection with FIG. 3.

At step 214A, the process can optionally obtain a video including a subject for a sequence length. In some examples, a subject can be a neonate. However, the subject is not limited to a neonate. The subject can be a non-neonate (e.g., baby, child, adult). In further examples, the sequence can indicate a group of data (e.g., frames/images, audio signals, etc.) in a sequence length or a predetermined period of time (e.g., 10 seconds, 20 seconds, 30 seconds, 1 minute, 10 minutes, 1 hour, 5 hours, 24 hours, or any other suitable period of time). In some embodiments, the duration of each sequence of data may overlap (e.g., a 30 second window overlaps by 20 seconds with the immediately prior window, a 10 second window overlaps by 5 seconds with the prior window, a 1 hour window overlaps by 59 minutes, 30 minutes, etc. with the prior window). The duration of each window may correspond to medical standards, such as the duration of monitoring for neonatal pain by medical professionals. In some embodiments, the process may dynamically alter the degree of overlap of the windows based upon frame rate and quality of the video acquisition, so that the process can be agnostic to the capabilities of the hardware used to acquire the video sequences. In certain embodiments, the video acquisition may be a constant feed, with windows been determined in real time as the video data stream is processed; whereas in other embodiments where processing, batter, or other resources may be constrained (or where a real time feed is not necessary), the sequences may arrive as individual files/packets. A video can include multiple frames or images in the sequence length.

At step 216A, the process can optionally preprocess the video data to generate multiple different types of data corresponding to different sensing modalities. For example, the process can extract visual (face and body) frames or images and extract audio data from the video. In some examples, the process can detect a facial region of a subject from the images (e.g., using an R-CNN or YOLO-based face detector, or other object detection algorithm trained to detect regions in an image or video stream that correspond to a face). In further examples, the process can detect the body region from the images (e.g., using another R-CNN or YOLO-based detector, or other object detection algorithm trained to detect regions in an image or video stream that correspond to a body). Then, the process can optionally resize all images/frames (e.g., 224×224) to provide a consistent data flow in the multimodal network. In some examples, facial images in the sequence length can be one modality, and body images in the sequence length can be another modality. In the case of the audio modality, the process can convert the audio data in the video stream to an isolated audio data stream with a predetermined sampling rate (e.g., 16K mono signals). In some scenarios, the process might not detect a face or a body in some images in the sequence due to the partial occlusion of the neonate's face or body. This may lead to a different number of frames belonging to face and body modalities. To fix this issue and remove repetitive frames, the process can extract the salient frames from these sequences with an equal time distribution. In some examples, the process can divide each sequence/window of video data into N equal segments. From each segment, F-number of frames can be chosen. In a non-limiting instance, the process can choose the value of N and F as 10 and 1, respectively; in other instances, N may be 30 and F may be 15, or other ratios. This frame selection can be random within equal length slots. For example, at first, N equal length segments can be chosen from the entire sample. Then randomly F frames can be chosen from each slot repeatedly during the training/testing phase. In some examples, the selection can be truly random or choose the best frames (e.g., select frames based on highest quality or pose, etc.). In some scenarios, the random selection of frames can be used in the training phase while specific frame selection can be used in the run-time phase. In other scenarios, the random selection of frames can be used in the training and run-time phases. In other scenarios, the specific frame selection can be used in the training and run-time phases.

At step 218A, the process can obtain multiple types of sensor data corresponding to one or multiple sensing modalities, for the sequences/windows for which video data was acquired in step 216A. (Note, steps 216A and 218A could occur simultaneously or in opposite order). For example, the process can receive multiple visual samples in the video, and each sample corresponding to the sequence can include n number of facial images and body images, i.e., Sⱼ=f₁, f₂, f₃, . . . , fₙ where Sⱼ∈S. For the auditory modality, Sⱼ can be just one audio signal. In further examples, sensor data for the face modality can include facial images of the subject (e.g., neonate) produced from the video for the sequence length (e.g., 10 seconds). Sensor data for the body modality can include body images of the subject produced from the video for the sequence length. Sensor data for the auditory modality can include audio data from the subject produced from the video for the sequence length. In further examples, sensor data is not limited to facial image, body image, an audio data. Sensor data can include other suitable time-series data for the sequence length. For example, sensor data for another modality can include vital signs (e.g., body temperature, blood pressure, respiration rate, pulse rate, etc.), environmental factors (e.g., ambient noise, temperature, room/scene lighting, humidity), and other states of a neonate such as wet/dry diaper, time since last feeding, etc. In other examples, the process can obtain multiple sensor data for multiple modalities without performing steps 216A and 218A. For example, the process can separately receive facial images, body images, and audio data for the sequence length from any suitable source (e.g., a server, a cloud, etc.). In some examples, the process can obtain sensor data of the multiple data modalities corresponding to the time period. In some examples, data of multiple data modalities can be determined base don the sensor data of the multiple data modalities and multiple sub-AI models. In some examples, the process can obtain the data of multiple data modalities corresponding to a time period. In some examples, the data of the multiple data modalities includes multiple intermediate feature corresponding to the multiple data modalities. For examples, the multiple intermediate features can be produced from multiple sub-AI models where the multiple intermediate features correspond to the multiple sub AI models (e.g., R-CNN or YOLO-based detector, FaceNet-based model, Google's VGGish model, etc.). The multiple data modalities can include at least one of a face modality, a body modality, or an auditory modality, and the sensor data of the plurality of data modalities can includes at least one of: multiple facial images for the face modality, multiple body images for the body modality, or audio data for the auditory modality.

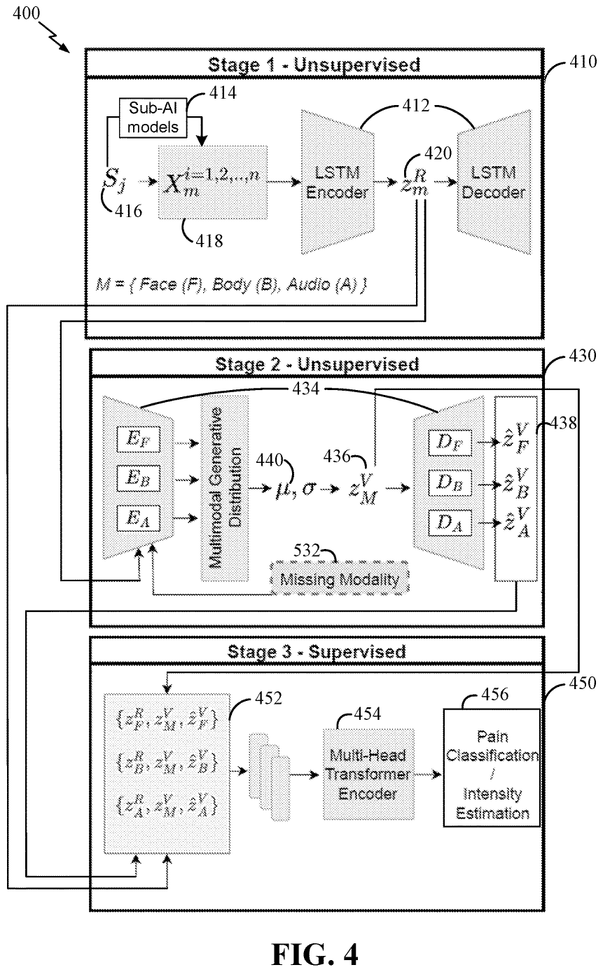

Steps 220A-228A describe certain three-stage pain assessment techniques based on AI models. FIG. 4 shows an example conceptual framework 400 for pain assessment according to some embodiments. In some examples, the framework 400 can include three stages 410, 430, 450 to generate a pain score of the subject. The first stage, second, and third stages 410, 430, 450 are described in steps 220A, 222A-226A, and 228A, respectively.

At step 220A, the process can determine a latent feature space 420 in the sequence for each modality 416 based on the trained first AI model 412, 414. For example, the first AI model 412, 414 can produce intermediate features (e.g., spatial/audio features) in the sequence/window length for each modality and produce a spatio-temporal latent space for each modality based on the intermediate features for a respective modality. Training the first AI model is further described below in connection with FIG. 3.

In some examples, the first AI model 412, 414 can include multiple sub-AI models 414 to capture one or more intermediate features 418 (e.g., spatial/audio features) of sensor data for each modality 416. For example, a first sub-AI model 414 corresponding to the face modality 416 can extract one or more spatial features 418 (i.e., intermediate features) from each facial image of the sensor data in the sequence length. In a non-limiting scenario, the first sub-AI model 414 can include a FaceNet-based model or any other suitable model (such as various types of CNNs) to extract one or more spatial features 418 for the facial modality 416. In further examples, a second sub-AI model 414 corresponding to the body modality 416 can extract one or more spatial features 418 (i.e., intermediate features) from each body image of the sensor data in the sequence length. In a non-limiting scenario, the second sub-AI model can include a Resnet18-based model or any other suitable model to extract the one or more spatial features 418 for the body modality 416. In even further examples, a third sub-AI model 414 corresponding to the auditory modality 416 can extract one or more audio features 418 (i.e., intermediate features) from each audio signal of the sensor data for the audio modality. In a non-limiting scenario, the third sub-AI model 414 can include a VGGish model or any other suitable model to extract one or more audio features 418 for the auditory modality 416. It should be appreciated that other sub-AI models 414 can be used to capture other features 418 of other modalities 416.

In further examples, the first AI model can further include an autoencoder neural network (e.g., long short-term memory (LSTM)-based autoencoder (AE)) 412. In some examples, the autoencoder neural network 412 can include two layers (e.g., an encoder and a decoder). It should be appreciated that the first AI model can be any other suitable neural networks (e.g., Hopfield, Boltzmann, RBM, Stacked Boltzmann, Helmholtz, etc.) trained in an unsupervised manner. In addition, the first AI model is not limited to unsupervised neural networks. The first AI model can include an artificial neural network (ANN), a convolutional neural network (CNN), a recurrent neural network (RNN), and any other suitable supervised neural networks.

In even further examples, the trained first AI model 412 (e.g., an LSTM-based autoencoder) can receive the one or more intermediate features 418 (spatial/audio features) of each modality 416. The trained first AI model 412 can generate a latent feature or latent feature space 420 (e.g., spatio-temporal latent space) for each modality 416. In some examples, the latent feature for each modality of the plurality of data modalities can include an output of an encoder in the autoencoder neural network. For example, an encoder (e.g., RNN encoder) of the first AI model 412 can receive the one or more intermediate features (i.e.,

$${X}_{m}^{i = 1 , 2 , … , n}$$

where m∈M: face modality (F), body modality (B), audio modality (A), i is indicative of an index of a frame in the sequence and n is indicative of the sequence length) for each modality 416 and generate a latent feature or latent feature space 420 (i.e.,

$${z}_{m}^{R}$$

where R represents RNN encoder/decoder) for each modality 416, which can be used in the second AI model 430 and the third AI model 450. Here, the latent feature or latent feature space 420 can be a spatio-temporal latent feature space or a fixed size latent feature space for each modality. Thus, the process can extract a latent feature space

$$( {z}_{m}^{R} )$$

for each modality (e.g., face, body, audio, etc.) based on the first AI model 410.

At step 222A, the process can determine if one or more modalities 432 are missing or one or more sensor data are missing in the sequence length. In some examples, a missing modality can indicate that entire sensor data corresponding the missing modality is missing or a substantial amount of the entire sensor data corresponding the missing modality is missing to consider the substantial amount as an input for the model. However, in other examples, a missing modality can include a part of the entire sensor data. For example, a neonate can be wrapped in blanket for the sequence length or a part of the sequence length in the video. Then, the whole or part of the sensor data for the body modality does not exist in the sequence length. In another example, a neonate does not face a camera for a few seconds or for the entire sequence length. Then, the process might not detect the face in frames/images for the few seconds or entire seconds during the sequence length and consider the frames or images as missing sensor data. In a further example, the neonate can be in a room with noise (e.g., music). Then, the process might not accurately detect the whole audio data or a part of the audio data in the sequence length from the neonate. In further examples, the process can determine the one or more missing modalities and/or missing senor data for a modality if a part of sensor data for a modality does not exist while other corresponding signals for other modalities in the timeline of the sequence are detected and exist. Once the process determines one or more missing modalities 432 and/or missing sensor data, the process can move to step 224A. If there is no missing modality, the process can move to step 228A.

At step 224A, the process can generate a common latent space 436

$$( {z}_{M}^{V} )$$

based on the trained second AI model 434 and the latent feature or latent feature spaces 420 for modalities. For example, the second AI model 434 can include a variational autoencoder (VAE) neural network (e.g., multilayer perceptron (MLP) encoder-decoder). In some examples, the second AI model can include four encoder layers (e.g., 128→128→128→64) and four decoder layers (e.g., 64→128→128→128) for each modality. However, it should be understood that any other suitable neural networks can be used for the second AI model 434. In further examples, the second AI model 434 can include a generative model (θ) and an inference model (φ). In some examples, the process can generate a probability distribution of the latent feature for each modality of the plurality of data modalities based on an inference model of the variational autoencoder neural network, and generate a joint-posterior distribution based on the probability distribution for each modality of the plurality of data modalities. In some examples, the common latent space can be generated based on the joint-posterior distribution. The process can estimate the probability distribution (μ, σ) 440 of the latent feature space 420 for each modality (F, B, A) using the parameterized inference model (φ). Then, the process can generate the common latent space 436

$$( {z}_{M}^{V} ,$$

where V represents the VAE model) based on the estimated probability distribution (μ, σ) 440. In some examples, the common latent space

$$( {z}_{M}^{V} )$$

436 can include a joint-posterior distribution. For example, the model is trained in such a way (combination of different modalities) so that when the modality is missing, the model knows how to generate the common latent space (e.g., using the concept of POE).

At step 226A, the process can generate a reconstructed latent space 438 for a missing modality based on the common latent space and the trained second AI model. In some examples, the reconstructed latent space 438 can be generated from an output of the decoder of the second AI mode. In further examples, each modality based on the common latent space 436 and the second AI model 434. For example, the process uses the decoder 434 of the second AI model 434 to generate the reconstructed latent space 438 for each modality or a missing modality based on the common latent space 436. In some examples, the encoder can encode and generate z, the decoder can receive z as input and generate \hat{z}, which is the reconstructed feature representing the encoder input. Thus, the reconstructed latent space 438 for each modality can include one or more missing modalities and/or missing sensor data of modalities.

At step 228A, the process can determine a pain indication and/or a level of intensity based on the multiple latent feature spaces, the common latent space, and the reconstructed latent spaces for the missing modality or the modalities, and the third trained AI model. In some examples, the process can stack the latent feature for each modality of the multiple data modalities and the reconstructed latent feature of the missing modality, generate multiple attentive features based on the trained third AI model and the stacked latent feature for each modality of the multiple data modalities with the reconstructed latent feature, and concatenate the multiple attentive features for a final feature vector where the pain indication can be determined based on the final feature vector. In some examples, the pain indication can include at least one of: a pain classification or a level of pain intensity. For example, after generating the latent feature space

$$( {z}_{m}^{R} )$$

and reconstructing missing modality

$$( {\hat{z}}_{m}^{V} )$$

from the common latent space

$$( {z}_{M}^{V} )$$

in steps at 220A (stage 1) and 224A and 226A (stage 2), the process can stack the latent features 452 of F, B, and A sensor data. In some examples, the process can use the trained third AI model to generate a pain indication (e.g., classification) and/or a level of intensity of the pain 456 based on the latent features 452. In some examples, the third AI model can include a transformer encoder 454. In further examples, the transformer encoder 454 can include a transformer encoder layer with 2 multi-heads to initially perform the scale-dot-product attention. In further examples, the process can apply an attentional fusion using the third AI model 454 as follows:

$$Attention ( Q , K , V ) = softmax ( \frac{Q {K}^{T}}{\sqrt{{d}_{k}}} ) V ,$$

where Q, K, V, and dₖ are the query, key, value matrix, and the scaling factor, respectively. Then, the process can select the latent feature space

$$( {z}_{M}^{R} )$$

or reconstructed latent space

$$( {\hat{z}}_{m}^{V} )$$

for each modality. z is the common generative space which represents combined features of all modalities, whereas \hat{z} is the individual modality features. \hat{z} can be used to analyze individual modality in the next stage. Then, the process can stack the selected features, and the third AI model can generate attentive features based on the selected features. The process can concatenate the attentive features and use the concatenated attentive features as a final feature vector. The process can determine the pain indication and the level of intensity based on the final feature vector. In some examples, a sigmoid function can be used for pain and no-pain classes. For the level of intensity, the third AI can include an MLP layer (e.g., following 384→256→128→Y). Y=1 can be a linear point for pain intensity estimation. In some scenarios, the third AI model can be trained as a regression problem. Then, the third AI model can estimate any continuous number in the range, for example, here, an example range (e.g., 0-7 and 0-4) can be reported. Thus, the third AI model can predict a continuous number within the range. In the scenarios, the system can additionally determine whether the pain exists when the continuous number is more than a predetermined threshold (e.g., 4 in 0-7 range or any other suitable threshold number). In other scenarios, the third AI model can be trained to produce a classification result (e.g., pain or no-pain).

In some examples, the process can constantly update the first, second, and third AI models based on the outputs of the first, second, and third AI models and post-administration effects or results, which are ground truth data. For example, the process can update the third AI model based on the predicted pain indication and the pain intensity level from the third AI model and the ground truth pain indication and intensity level determined by a medical practitioner.

FIG. 2B is a flow diagram illustrating another example process 200B for pain assessment in accordance with some aspects of the present disclosure. As described below, a particular implementation can omit some or all illustrated features/steps, may be implemented in some embodiments in a different order, and may not require some illustrated features to implement all embodiments. In some examples, an apparatus (e.g., computing device 110) in connection with FIG. 1 can be used to perform the example process 200B. However, it should be appreciated that any suitable apparatus or means for carrying out the operations or features described below may perform the process 200B. The process 200B is generally directed to a runtime stage using one or more trained artificial intelligence (AI) models. Training the AI models is described in connection with FIG. 3. In a non-limiting scenario, the process 200B can be used for postoperative pain assessment and/or intensity. However, the process 200B can be used for any other suitable purposes (e.g., preoperative pain assessment, pain prediction, etc.).

At step 212B, the process can obtain a trained first artificial intelligence (AI) model, a trained second AI model, and a trained third AI model. Step 212B is substantially similar to step 212A in FIG. 2A.

At step 214B, the process can obtain data of multiple data modalities corresponding to a time period. In some examples, the data of the multiple data modalities can include multiple intermediate features corresponding to the multiple data modalities. In some examples, the multiple intermediate features can be produced from multiple sub-AI models where the multiple intermediate features correspond to the multiple sub-AI models. In some examples, the process can obtain sensor data of the plurality of data modalities corresponding to the time period, and the data of the plurality of data modalities can be determined based on the sensor data of the plurality of data modalities and the plurality of sub-AI models. In some examples, the multiple data modalities can include at least one of: a face modality, a body modality, or an auditory modality. Further, the sensor data of the plurality of data modalities can include at least one of: a plurality of facial images for the face modality, a plurality of body images for the body modality, or audio data for the auditory modality. Step 214B is substantially similar to step 214A, 214A, and/or 216A in FIG. 2A.

At step 216B, the process can generate a latent feature of the data for each modality of the plurality of data modalities based on the trained first AI model and the data for each modality of the plurality of data modalities. In some examples, the trained first AI model can include an autoencoder neural network. Further, the latent feature for each modality of the plurality of data modalities comprises an output of an encoder in the autoencoder neural network. Step 216B is substantially similar to step 220A in FIG. 2A.

At step 218B, the process can generate a common latent space based on the trained second AI model and the latent feature space of each modality of the plurality of data modalities. In some examples, the trained second AI model comprises a variational autoencoder neural network. In some examples, the process can further generate a probability distribution of the latent feature for each modality of the plurality of data modalities based on an inference model of the variational autoencoder neural network, and generate a joint-posterior distribution based on the probability distribution for each modality of the plurality of data modalities. The common latent space can be generated based on the joint-posterior distribution. Step 218B is substantially similar to step 224A in FIG. 2A.

At step 220B, the process can generate a reconstructed latent feature for a missing modality based on the common latent space and the trained second AI model. In some examples, a decoder or generative model of the trained second AI model can produce the reconstructed latent feature for the missing modality based on the common latent space. Step 220B is substantially similar to step 226A in FIG. 2A.

At step 222B, the process can determine a pain indication based on the latent feature for each modality of the plurality of data modalities, the reconstructed latent feature for the missing modality, and the trained third AI model. In some examples, the trained third AI model can include a transformer encoder neural network. In further examples, the process can further stack the latent feature for each modality of the plurality of data modalities and the reconstructed latent feature of the missing modality, generate multiple attentive features based on the trained third AI model and the stacked latent feature for each modality of the plurality of data modalities with the reconstructed latent feature, and concatenate the multiple attentive features for a final feature vector. In some examples, the pain indication can be determined based on the final feature vector. Further, the pain indication can include at least one of: a pain classification or a level of pain intensity. Step 222B is substantially similar to step 228A in FIG. 2A.

In further example, the process can obtain a vital sign and produce a sepsis indication based on the pain indication and the vital sign. In some examples, the vital sign can include at least one of: a body temperature, a pulse rate, a respiration rate, or a blood pressure. In some examples, an early sign of sepsis can be diagnosed based on a body temperature (e.g., being higher than 100.4 °F), a pulse rate (e.g., being 90 beats per minute), and/or a respiration rate (e.g., being greater than 20 breaths per minute) with/without a low blood pressure. In addition to the vital sign, a pain indication (e.g., a pain score being greater than a threshold pain score) can increase the accuracy to predict the sepsis. In some example, the sepsis indication can include a level or a stage of severity of sepsis, a classification of sepsis diagnoses (e.g., yes or no) or any other suitable indications. In other embodiments, a prediction of sepsis and/or a classification of sepsis severity can be made in parallel with the monitoring of a patient as described herein. For example, heart rate variability (HRV) can be utilized to assess severity of illness, poor outcomes, and mortality in patients having sepsis or suspected of having sepsis. Thus, in some embodiments, a system for monitoring a patient for purposes of pain assessment and prediction (as described herein) can be augmented by monitoring a patient' vital signs. An algorithm may be running in real time that receives signals reflecting a patient's vital signs. If that algorithm makes a sepsis determination, the system can generate an alert to the patient's caregiver via the same medical records system and/or patient monitor display that is used to normally communicate with the care team. This determination of sepsis may also be utilized as an input to the model that makes pain predictions and pain medication suggestions. In such circumstances, if a patient is in a severe state of sepsis or is predicted to be in a state of sepsis, the model may forego suggesting a pain medication dosage. For example, the system may thereby avoid a scenario in which a patient with sepsis has a delayed diagnosis or treatment due to symptoms being masked by pain medication.

FIG. 3 is a flow diagram illustrating an example process for pain assessment system training according to some embodiments. As described below, a particular implementation can omit some or all illustrated features and may not require some illustrated features to implement all embodiments. In some examples, an apparatus (e.g., computing device 110) in connection with FIG. 1 can be used to perform the example process 300. However, it should be appreciated that any suitable apparatus or means for carrying out the operations or features described below may perform the process 300. The process 300 is generally directed to a training stage of a first AI model and a second AI model for pain assessment.

Steps 312-316 are substantially the same as steps 214A-218A, respectively. For example, at step 312, the process can obtain a video including a subject for a sequence length. At step 314, the process can preprocess the video to generate multiple sensor data for corresponding modalities. In some examples, steps 312 and 314 can be optional. At step 316, the process can obtain multiple sensor data for corresponding modalities for the sequence length. For example, the process can obtain sensor data (e.g., facial images) for a face modality, sensor data (body images) for a body modality, and sensor data (audio data) for an auditory modality for the sequence length. In some examples, sensor data is not limited to facial image, body image, an audio data. Sensor data can include other suitable time-series data for the sequence length. For example, sensor data for another modality can include vital signs (e.g., body temperature, blood pressure, respiration rate, pulse rate, etc.). In further examples, the process can perform augmentation of the sensor data by random rotation (±30) and horizontal flip. However, it should be appreciated that the sensor data augmentation is not limited to random rotation and horizontal flip. It could include scaling, translation, cropping, adding noise, contrast, saturation, brightness, etc. This augmentation can be applied to all frames of a particular sequence dynamically during the training time. In some examples, to obtain the training data of multiple data modalities, the process can obtain unfiltered data of the plurality of data modalities corresponding to the time period; and selecting the training data among the unfiltered data by an influence score of each of the training data being equal or higher than a threshold score.

In some examples, the process can calculate the influence score for each of the training data based on an equation defined by:

$${{I}_{u p , l o s s} ( z , {z}_{t e s t} ) {=}^{def} \frac{d L ( {z}_{t est} , {\hat{θ}}_{ϵ , z} )}{d ϵ} \vert}_{ϵ = 0} = - ∇ {L ( {z}_{test} , \hat{θ} )}^{T} {H}_{\hat{θ}}^{- 1} {∇}_{θ} L ( z , \hat{θ} ) ,$$

where z is a training instance,

$$\hat{θ} {=}^{def} arg {min}_{{θ}_{ϵ Θ}} \frac{1}{n} {\sum }_{i = 1}^{n} L ( {z}_{i} , θ ) .$$

Thus, the process can train the first, second, and third AI models with fewer training data by dropping harmful training data without decreasing the accuracy. These harmful training data or low influence scored training data can be removed to create a more compact dataset without degrading the model's performance.

At step 318, the process can train or obtain multiple sub-AI models corresponding to the multiple modalities to extract intermediate features of sensor data of each modality. In some examples, the first AI model 412, 414 can include multiple sub-AI models 414 to extract features (e.g., intermediate features 418 of FIG. 4) from the sensor data of multiple corresponding modalities. In some examples, the process can train a first sub-AI model 414 corresponding to the face modality 416 to extract one or more spatial features 418 (i.e., intermediate features) from each facial image of the sensor data in the sequence length. In other examples, the process can obtain a pre-trained first sub-AI model 414 (e.g., pre-trained on the VGGFace2 dataset) to extract one or more spatial features 418 of a facial image. In some instances, the spatial features 418 in the sequence length for the face modality can include a spatial feature vector. In a non-limiting scenario, the first sub-AI model 414 can include a FaceNet-based model or any other suitable model to extract spatial features 418 for the facial modality 416.

In further examples, the process can train a second sub-AI model 414 corresponding to the body modality 416 to extract one or more spatial features 418 (i.e., intermediate features) from each body image of the sensor data in the sequence length. In other examples, the process can obtain a pre-trained second sub-AI model 414 (e.g., pre-trained on the ImageNet dataset). In some instances, the spatial features 418 in the sequence length for the body modality can include another spatial feature vector. In a non-limiting scenario, the second sub-AI model can include a Resnet18-based model or any other suitable model to extract spatial features 418 for the body modality 416.

In even further examples, the process can train a third sub-AI model 414 corresponding to the auditory modality 416 to extract one or more audio features 418 (i.e., intermediate features) from each audio signal of the sensor data for the audio modality. In other examples, the process can obtain a pre-trained third sub-AI model 414 (e.g., pre-trained with YouTube-8M dataset) to extract one or more audio features 418. In a non-limiting scenario, the third sub-AI model 414 can include a VGGish model or any other suitable model to extract one or more audio features 418 for the auditory modality 416. It should be appreciated that other sub-AI models 414 can be used to capture other features 418 of other modalities 416.

At step 320, the process can train the first AI model in an unsupervised manner based on the intermediate features

$$( {X}_{m}^{i = 1 , 2 , … , n} )$$

418 of the sensor data of each modality. The intermediate features obtained at step 318 can be used to train the first AI model 412 (e.g., LSTM-based AE as noted at step 220A of FIG. 2) in an unsupervised manner, where the encoder learns a compressed spatio-temporal feature representation from the deep features. For an intermediate feature

$${X}_{m}^{i}$$

with dₘ feature-length and n sequence length, the first AI model can map the sequence as follows:

$${E}_{R} : {X}_{m}^{i = 1 , 2 , … , n} \to {z}_{m}^{R} and {D}_{R} : {z}_{m}^{R} \to {\hat{X}}_{m}^{i = 1 , 2 , … , n} ,$$

where m∈M, E and D are the RNN encoder and decoder functions of the first AI model, respectively,

$${z}_{m}^{R}$$

is the fixed size latent feature space of the first AI model (e.g., RNN AE), and \hat{X} are the reconstructed features. Based on the reconstructed features (\hat{X}) and the input intermediate features (X), the process can calculate the loss function (L_{R}) using the mean square error (MSE) as follows:

$${L}_{R} = \frac{1}{n} {\sum }_{i = 1}^{n} {( {X}_{m}^{i} - {\hat{X}}_{m}^{i} )}^{2} .$$

Based on the loss function, the process can train the first AI model to learn the feature reconstruction. It should be appreciated that the first AI model is not limited to unsupervised training. In some examples, the first AI model can be trained in a supervised manner with given ground truth data.

At step 322, the process can train the second AI model 434 in an unsupervised manner based on the latent feature spaces 420 from the first AI model 412 of each modality. For example, the process can train the second AI model 434 using a loss function based on a common latent feature space

$${z}_{M}^{R} .$$

and a latent feature

$$( i . e . , {z}_{F}^{R} , {z}_{B}^{R} , and {z}_{A}^{R} )$$

for each modality generated by the first AI model 412. In some examples, the second AI model 434 can be trained with the trained first AI model 412. Thus, after training the first AI model 412, the process can extract the latent feature space or vector

$${z}_{m}^{R}$$

420 for each modality, and the second AI model 434 can generate a common latent feature space 436 based on the latent feature space 420 of each modality. The second AI model 434 can include a VAE to learn the joint probability distribution of the vectors. In a non-limiting scenario, the VAE can include a generative model (θ) and an inference model (φ), and the VAE can be optimized through Evidence Lower Bound (ELBO). The process can estimate the probability distribution (μ, σ) 440 of the latent feature space 420 for each modality (F, B, A) using the parameterized inference model (φ).

In some examples, the process can use a product of expert approximation (POE) to generate a joint-posterior distribution. The POE can act as a common parameterized inference model to estimate the final probability distribution of the joint latent space. ELBO can be defined based on the combination of the likelihood and Kullback-Leibler (KL) divergence as follows:

$$ELBO ( {z}_{m}^{R} ) := {𝔼}_{q ϕ \vert {z}_{m}^{R}} [ λ log {p}_{θ} ( {z}_{m}^{R} \vert {z}^{V} ) ] - β KL [ {q}_{ϕ} ( {z}^{V} \vert {z}_{m}^{R} ) , p ( {z}^{V} ) ] ,$$

where

$${z}_{M}^{R}$$

and z^{v} are the observation and the latent space, respectively;

$${p}_{θ} ( {z}_{m}^{R} \vert {z}^{V} )$$

and

$${q}_{ϕ} ( {z}^{V} \vert {z}_{m}^{R} )$$

are the generative model and inference model respectively; p(z^{V}) is the prior; λ and β can be the controlled parameters. The process can incorporate the POE over multiple sensor data of multiple modalities as follows:

$$ELBO ( {z}_{m}^{R} ) := {𝔼}_{q ϕ \vert {z}_{m}^{R}} [ {\sum }_{m ∈ M} {λ}_{m} log {p}_{θ} ( {z}_{m}^{R} \vert {z}^{V} ) ] - β KL [ {q}_{ϕ} ( {z}^{V} \vert {z}_{m}^{R} ) , p ( {z}^{V} ) ] .$$

In some examples, the process can optimize ELBO of the joint signals instead of individual signals. In further examples, the process can pass Null values for the ELBO of the individual signals, and can define the joint learning loss (Lv) from the second AI model as follows:

$${L}_{V} = ELBO ( {z}_{M}^{R} ) + ELBO ( {z}_{F}^{R} ) + ELBO ( {z}_{B}^{R} ) + ELBO ( {z}_{A}^{R} ) .$$

Thus, the second AI model 434 can be trained under different missing data conditions based on a common latent feature space

$${z}_{M}^{R}$$

436 and a latent feature

$$( i . e . , {z}_{F}^{R} , {z}_{B}^{R} , and {z}_{A}^{R} ,$$

one or more of the latent features can be null value for training) for each modality generated by the first AI model 412. Specifically, if any sensor data is missing, the second AI model (e.g., POE) can create the generative probability distribution, which is used to generate the common latent features

$$( {z}_{M}^{R} )$$

that acts as a common joint feature for all signals. In some examples, the process can use MSE as the loss function. It should be appreciated that the second AI model is not limited to unsupervised training. The second AI model can be trained in a supervised manner with given ground truth data.

At step 324, the process can train a third AI model 454 in a supervised manner based on the multiple latent feature spaces 420 from the first AI model 412, a common latent space 436, and multiple reconstructed latent spaces 438 for modalities from the second AI model 434. In some examples, when there is no missing modality or sensor data, the process can receive the multiple latent feature spaces 420 from the first AI model 412. In other examples, the third AI model 454 can receive the multiple latent feature spaces 420 from the first AI model 412, a common latent space 436, and multiple reconstructed latent spaces 438 for modalities from the second AI model 434. Then, the process can stack the incorporated latent spaces 452 to input the stacked latent spaces 452 to the third AI model 454. For example, the incorporated latent spaces 438 can include a matrix. The matrix can include a row for each modality and three columns for a latent space

$${z}_{m}^{R}$$

420 from the first AI model 412, a common latent space

$${z}_{M}^{V}$$

436 from the second AI model 434, and a reconstructed latent space

$${\hat{z}}_{m}^{V}$$

438 from the second AI model 434. For example, a row of the matrix for the face modality can include the latent space

$$( {z}_{F}^{R}$$

420 for the face modality, the common latent space

$${z}_{M}^{V}$$

and the reconstructed latent space

$${\hat{z}}_{F}^{V}$$

for the face modality, which can be expressed, in some scenarios, as follows:

$$\{ {z}_{F}^{R} , {z}_{M}^{V} , {\hat{z}}_{F}^{V} \} .$$

Similarly, other rows for the body modality and the auditory modality in the matrix can be expressed as follows:

$$\{ {z}_{B}^{R} , {z}_{M}^{V} , {\hat{z}}_{B}^{V} \}$$

and

$$\{ {z}_{A}^{R} , {z}_{M}^{V} , {\hat{z}}_{A}^{V} \}$$

respectively. It should be appreciated that other rows can exist for other suitable modalities.

In some examples, the third AI model 454 can receive the incorporated latent spaces 452. In some examples, the third AI model 454 can include a transformer encoder 454. In further examples, the transformer encoder 454 can include a transformer encoder layer with 2 multi-heads to initially perform the scale-dot-product attention. In further examples, the process can apply an attentional fusion using the third AI model 454 as follows:

$$Attention ( Q , K , V ) = soft max ( \frac{{QK}^{T}}{\sqrt{{d}_{k}}} ) V ,$$

where Q, K, V, and dₖ are the query, key, value matrix, and the scaling factor, respectively. Then the process can select the latent feature space

$$( {z}_{m}^{R} )$$

or reconstructed latent space

$$( {\hat{z}}_{m}^{V} )$$

for each modality. Then, the process can stack the selected features, and the third AI model can generate attentive features based on the selected features. The process can concatenate the attentive features and use the concatenated attentive features as a final feature vector. The process can determine the pain indication and the level of intensity based on the final feature vector. In some examples, a sigmoid function can be used for pain and no-pain classes. For the level of intensity, the third AI can include an MLP layer (e.g., following 384→256→128→Y). Y=1 can be a linear point for pain intensity estimation.

In some examples, the process can train the third AI model 454 based on the predicted result (the pain/no-pain class and/or the pain intensity level) and a ground truth label (the pain/no-pain class and/or the pain intensity level). In some examples, an individual ground truth label can be provided based on the observation of the entire modality, not per frame. In further examples, a final ground truth can be provided based on all sensor data. This final ground truth can be indicative of pain or no-pain along with an intensity score. In some examples, the final ground truth can include a pain classification indication to indicate the binary classification of pain and no-pain. The pain classification indication can include a bit, a number, a letter, a symbol, or any other suitable indication. In further examples, the final ground truth can further include a pain intensity level to indicate a level the pain intensity. The pain intensity level can include a byte, a letter, a symbol, or any other suitable indication.

The evaluation of the disclosed example approach (three stages) and the performance of both pain classification and intensity estimation is presented. The accuracy, F-1 score, area under the receiver operating characteristic curve (AUC) was used to report the performance of binary classification and mean squared error (MSE) and mean absolute error (MAE) to report the performance of intensity estimation. All the models were developed based on PyTorch environment using a GPU machine (Intel core i7-7700K@4.20 GHz, 32 GB RAM, and NVIDIA® GV100 TITAN V 12 GB GPU).

Dataset: The University of South Florida Multimodal Neonatal Pain Assessment Dataset (USF-MNPAD-I) neonatal pain dataset was used, which is the only publicly available neonatal postoperative pain dataset for research use. This dataset has 36 subjects recorded during acute procedural pain, and 9 subjects during postoperative pain. Each subject has videos (face and body) and audios (crying and background noises) recorded in the neonatal intensive care unit (NICU) of a local hospital. Each video and audio contain pain and no-pain segments that are labeled with two manual pain scales: neonatal infant pain scale (NIPS) scale for procedural pain and neonatal pain, agitation, and sedation (N-PASS) scale for postoperative pain. The procedural part of the dataset was used to learn the spatio-temporal features. The postoperative part was used to learn the joint feature distribution and reconstruct the missing modalities.

TABLE 1
Performance of the following approach and previous
works when all signals are present.

| Approach | Accuracy | Precision | Recall | F1-score | TPR | FPR | AUC |
|---|---|---|---|---|---|---|---|
| CNN-LSTM | 0.7895 | 0.7913 | 0.7895 | 0.7863 | 0.8761 | 0.3243 | 0.8791 |
| EmbraceNet | 0.7921 | 0.7919 | 0.7921 | 0.7920 | 0.8182 | 0.2405 | 0.8790 |
| Disclosed | 0.8230 | 0.8230 | 0.8202 | 0.8207 | 0.8080 | 0.1646 | 0.9055 |

Network Architectures and Training: In Stage 1, the state-of-the-art models were used to extract spatio-temporal feature vectors with 512-d, 512-d, and 128-d length from F, B, A signals, respectively. For temporal learning, an individual long short-term memory autoencoder (LSTM AE) with 2 layers was used, taking the respective spatial feature vector of input sequences to produce a spatio-temporal 128-d latent space. As mentioned above, the video has a sequence length of ≈10 s. In Stage 2, the multilayer perceptron (MLP) encoder-decoder following 128→128→64 and 64→128→128→128 encoder and decoder layers for each sensory signal was used. In Stage 3, a transformer encoder layer with 2 multi-heads had been used to initially perform the scale-dot-product attention. After that, all the features were concatenated (128+128+128=384). Next, an MLP layer following 384→256→128→Y was used. In case of binary classification, a sigmoid function was used for pain and no-pain classes. As for estimation, Y=1 is just a linear point for pain intensity estimation. A total of 218 postoperative videos (50% pain) were included in the following experiments. Following previous approaches, a leave-onesubject-out (LOSO) evaluation was performed. For the spatio-temporal training, the procedural dataset to learn the spatio-temporal features until convergence was used. For recurrent neural networks (RNN) autoencoder, Adam optimizer with 0.001 learning rate and 16 batch size was used. In the joint learning and attentional feature learning, LOSO and used Adam optimizer with 0.0001 learning rate and batch size of 8 was followed.

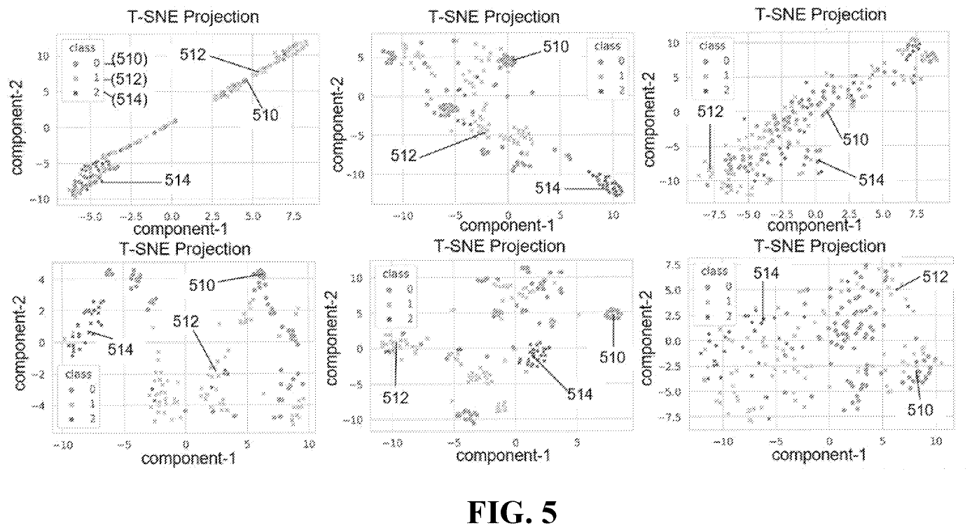

Visualization of Spatio-temporal Features: Spatio-temporal features were computed using FaceNet (face), ResNet18 (body), and VGGish (sound). To evaluate the quality of the extracted features, the t-SNE projections for all modalities was generated, as shown in FIG. 5. In some examples, all modalities are trained on the procedural pain set (unsupervised) and tested on the postoperative set. From FIG. 5, the feature points are scattered in the first row, which shows the baselines for face, body, and sound is observed. The baseline for face and body signals are the raw pixels obtained from the video modality while the baseline for the sound is the mel frequency cepstral coefficients (MFCCs) calculated from the auditory modality. On the contrary, the second row shows the feature points, which are generated by stage 1, grouped into clusters indicating a good differentiation capability of the extracted features.

TABLE 2
Performance of the proposed approach and when dropping each modality.

| | | | | F1- | | | |
|---|---|---|---|---|---|---|---|
| Approach | Modalities | Reconstruction? | Accuracy | score | TPR | FPR | AUC |
| CNN-LSTM | DropFace | No | 0.7719 | 0.7522 | 0.9897 | 0.5135 | 0.8763 |
| | DropBody | No | 0.6901 | 0.6703 | 0.8866 | 0.5676 | 0.8396 |
| | DropSound | No | 0.7076 | 0.6630 | 1.0000 | 0.6757 | 0.8353 |
| Disclosed | DropFace | Yes | 0.7921 | 0.7928 | 0.7576 | 0.1646 | 0.9022 |
| | DropBody | Yes | 0.8258 | 0.8257 | 0.8485 | 0.2025 | 0.9086 |
| | DropSound | Yes | 0.6854 | 0.6374 | 0.9899 | 0.6962 | 0.8028 |

TABLE 3
Ablation study of the attentional feature fusion.

| Approach | Accuracy | Precision | Recall | F1-Score | TPR | FPR | AUC |
|---|---|---|---|---|---|---|---|
| ST + JF | 0.5229 | 0.7559 | 0.5229 | 0.3824 | 0.9999 | 0.9541 | 0.5757 |
| ST + JF + AF | 0.7890 | 0.7899 | 0.7890 | 0.7888 | 0.7615 | 0.1835 | 0.8870 |

* ST = Spatio-Temporal, JF = Joint Features, AF = Attentional Fusion

Pain Assessment without and with Missing Modalities: The disclosed example classifier was compared with convolutional neural networks-long short-term memory (CNN-LSTM) approach and another multimodal approach named EmbraceNet. In this experiment, pain assessment in a subset of USF-MNPAD-I that has all the sensory signals present (F, B, A) was performed. From Table 1, the disclosed example approach outperformed and achieved 0.820 accuracy and 0.906 AUC is observed. Although the approach outlined achieved a lower true positive rate (TPR) as compared to the existing CNN-LSTM, it improved the false positive rate (FPR) (0.165) by almost 50%. Similarly, this approach significantly outperformed EmbraceNet (p<0.01). To evaluate the performance of the following approach and the novel reconstruction method, each sensory signal was completely dropped (100%), the features of the dropped signal were reconstructed, combined with the features of other signals, and the performance of multimodal pain classification was reported. The pain assessment performance using CNN-LSTM was also reported, as it is the most recent work in the literature that uses USF-MNPAD-I dataset. It is noted the existing CNN-LSTM discarded missing modalities when making a final assessment. It is duly considered that missing a sensory signal is common in clinical practices due to several factors including sensor failure, swaddling, or intubation, among others. The following model can classify any case with missing modalities as it can reconstruct these modalities and integrate them into the assessment. From Table 2, it is observed that reconstructing the features of face and body using the following approach improved the performance as compared to CNN-LSTM. The lower performance of sound suggests that sound reconstruction has a higher impact on the final pain/no-pain decision, which is consistent with a similar trend observed in Salekin's previous work.

Multimodal Assessment with Attentional Feature Fusion: Unlike other approaches, an attentional fusion to examine the cross-modal influence on the decision was used. To evaluate this fusion approach, an ablation study was performed, in which the performance of pain classification with and without attentional fusion was reported. In Table 3, it is observed that the proposed attentional fusion (ST+JF+AF) improved the pain classification performance by a large margin, demonstrating the effectiveness of this fusion approach.

As the pain intensity in USF-MNPAD-I dataset ranges from 0 to 7, a regression-based training to generate the intensity score was performed. An MSE of 3.95 and an MAE of 1.73 was found, which are reasonable for this relatively small and challenging dataset. The intensity range was further minimized and found better results which are 0-4 (MSE 0.75, MAE 0.73) and 0-1 (MSE 0.13, MAE 0.27). It was also found that the proposed approach is capable of understanding the no-pain/pain/no-pain transitions while estimating pain intensity with a success rate of 71.15%.

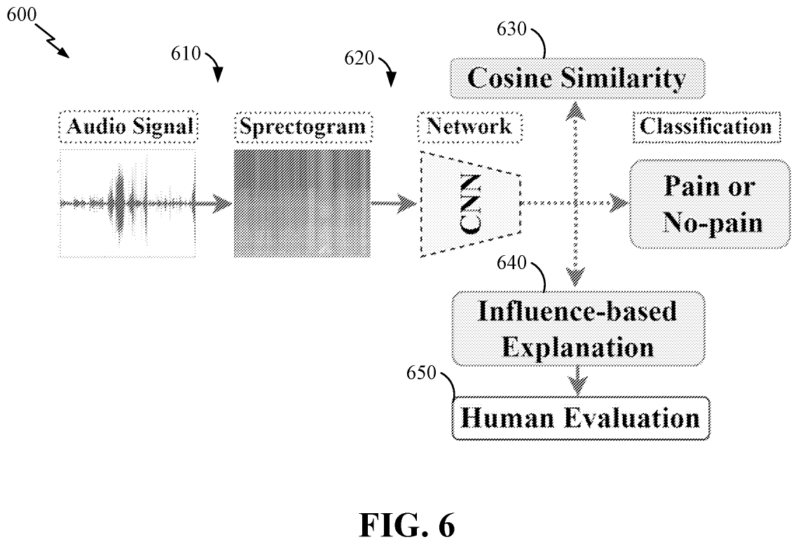

FIG. 6 is an example of an embodiment in which an influence function-based method is integrated into a pain classification model. In some examples, an influence-based approach for explaining the output of a model as described herein for estimating and/or predicting pain may be helpful for health care staff to have greater confidence in the output of the model, and promotes human involvement before making important decisions about healthcare. In some embodiments, a embodiment may provide an output (e.g., a pain score or a pain prediction) as described above, together with an influence-based explanation. For example, a model may output a pain score via a patient monitor display screen, and at the same time display on that screen that the score is heavily based upon a crying sound analysis. This not only improves transparency and explainability of the pain classification/prediction model, but helps an involved healthcare individual ascertain whether an error may have occurred (e.g., the patient is not crying, but a similar sound was present, or an undetected external factor caused brief crying). For example, in some instances, a model may predict future pain and suggest a dosage of a pain medication. However, the interface used by the healthcare provided may require them to first verify that the factors influencing the prediction appear accurate (e.g., crying was actually detected, no soiled diaper, etc.) before the system will dispense the suggested quantity of pain medication. Thus, the patient's medical record will reflect a consensus of both the human caregiver and the model.

In alternative embodiments, the human caregiver may disagree with the model and intervene by inputting an indication that the factors influencing a given pain prediction are not actually present or are not a cause of pain. In this case, the system may be programmed to continue monitoring the patient and determine whether the model's prediction was correct or whether the human caregiver's intervention was correct. If the model's prediction was incorrect, then a notice may be sent to developers responsible for the model to indicate that a new potential training case should be utilized to re-train the model or that other de-bugging should take place. If the model's prediction was correct, then the system may automatically update the caregiver.

Thus, after obtaining the pain indication from the process of FIG. 2A or 2B, this influence-based explainable artificial intelligence (XAI) method may be utilized to leverage an understanding of the reasoning behind the model's assessment or prediction. Although the example influence function-based method described above is based on a crying sound, the example influence function-based method can be used any other modality (e.g., face modality, body modality, etc.).

In some examples, the first step 610 can involve converting the original audio signal into spectrogram images followed by the second step 620 to send the spectrogram images to the CNN for pain classification/estimation/detection. Then, the cosine similarity 630 and influence scores 640 can be calculated between the test and train images. Finally, the outcome of our explanation method is assessed by a human evaluator 650.

Data Preparation and Augmentation: In some examples, the audio signals can be extracted from videos of infants experiencing postoperative pain. Then the extracted signals can be converted into a frequency representation image known as the spectrogram image. Spectrogram images can lead to better performance as spectrogram images can suppress the noise in the audio signal.

As the dataset has a limited number of crying sound segments (218 segments), the dataset can be enlarged as follows. Each raw audio signal can be augmented by altering the fundamental frequency f at three levels (f/3, f/2, 2f/3), adding six various levels of noise (0.001, 0.003, 0.005, 0.01, 0.03, 0.05), and combining both noise and frequency; e.g., f/3 with noise 0.003 or f/3 with noise 0.005. The augmentation generates a total of 27 segments per audio signal; i.e., 3 for frequency variation, 6 for noise addition, and 18 (3×6) for the combination of frequency and noise. The augmented signals are then converted to spectrogram images. These images are used to fine-tune a pre-trained (ImageNet weights) VGG-16 architecture.

Responsible Training Instances Identification: The influence function is used to explain a given pain segment by identifying the most important training instances that impact the model's performance. Algorithm 1 details the steps of using the influence function to identify the responsible training instances. To calculate the influence score I_{up,loss}(z,zₜₑₛₜ) that each training instance has on the model's prediction for the test instance zₜₑₛₜ, this equation:

$${I}_{upmloss} ( z , {z}_{test} ) {=}^{def} \frac{dL ( {z}_{test} , {\hat{θ}}_{ϵ , z} )}{d ϵ} {\vert}_{ϵ = 0} = - ∇ {L ( {z}_{test} , \hat{θ} )}^{T} {H}_{\hat{θ}}^{- 1} {∇}_{θ} L ( z , \hat{θ} )$$

is used. This enables to determine which training instance has the most positive or negative influence on that test instance; the most helpful training instance has the highest influence score, and the most harmful training instance has the lowest score.

Algorithm 1: Audio Modality Explanation

```
Input: Dataset S, Training data zᵢ, Test data zₜ
Output: Influence Score
Procedure KeyTrainData (S, zᵢ, zₜₑₛₜ)
  for each zᵢ ∈ S do
    Calculate influence I_{up, loss} (zₜₑₛₜ, z) using the equation above
    Obtain influence score for the training data (zᵢ)
    Sort and identify the training data as helpful/harmful for the test data zₜₑₛₜ
  end
  for each zᵢ ∈ S do
    Calculate the cosine similarity using cos(φ(zₜₑₛₜ), φ(z))
    Play each test instance zₜₑₛₜ and perform the human evaluation
  end
return Influence score, responsible data index
```

To distinguish between the training instances that improve the model's performance and those that degrade the performance, various percentages of the harmful and helpful training instances can be further dropped. Finally, a human-grounded evaluation is applied to verify whether the influence function produced the expected results; i.e., a trained graduate student evaluated the results, which were further verified by a senior researcher in our lab. The mapping to the high-dimensional feature space results in a dense feature vector, where each vector dimension has a non-zero value. There are several methods (e.g., CNN-based, autoencoder-based, hand-crafted feature-based, and transformer-based) for obtaining a dense vector representation.

In this work, a CNN-based method is used as the embeddings obtained through CNNs are rich in spatial and semantic information. Specifically, the last layer of a VGG-16 model is selected to reduce the image dimension while keeping the most important information. The final layer of VGG-16 provides a feature vector #(z) of 512 values. Once the feature vectors is given for the training (φ(zᵢ)) and test images (φ(zₜ)), the cosine similarity of images can be computed as follows: cosₛᵢₘ=cos(φ(zₜ), φ(zᵢ)). The similarity measurement is 1 or close to 1 when the test image is identical or very similar to the training image. Finally, the obtained similarity score is contrasted with the influence score to evaluate the outcome of our approach. If the test image is similar to a specific training image, the influence score of that training image should be high; i.e., the model's performance will degrade significantly when that training image is removed.

In some examples, the processes 200A, 200B, and/or 300 can be used for automatic and real-time neonatal postoperative pain assessment. The processes 200A, 200B, and/or 300 are proven to be effective in constructing and accounting for missing data/signals, which is a common situation in neonatal intentional care unit (NICU) settings. In addition, the processes 200A, 200B and/or 300 are proven to be effective in enhancing multimodal pain assessment. In other words, more robust and more accurate assessments can be achieved through use of multiple modalities of sensing (facial expression, body movement, environmental factors, vital signs, etc.)

Given that a robust and accurate pain assessment can be automatically made using the techniques provided herein, further advances can be made in terms of employing systems to help manage and predict pain treatment for subjects. In some embodiments, a prediction can be made of future pain intensity, based upon measurements made of a subject (e.g., existing pain intensity) over time elapsed since a given even such as post-surgery/post-operation, and taking into account time since last pain medication delivery. In further embodiments, the method can be used to provide a continuous pain prediction signal. The only difference here is during training, input-output will be current-future. If the signal crosses the pain threshold (clinical practice range) for a certain amount of time (window prediction), it can alert the system to take necessary steps to control the pain and bring it back to the below threshold. For example, an algorithm may be trained to predict when a given level of pain intensity will be experienced by a neonate, based upon time-series measurements of pain intensity from other, similar subjects in similar situations. In a different non-limiting scenario, the third AI model can be trained with sensor data at time i and a ground truth data at time i+l (e.g., a future time) or at multiple future times i+l₁, i+l₂, . . . i+lₙ, wherein each increment l represents a future length of time period (e.g., 10 seconds, 30 seconds, 1 minute, 5 minutes, 10 minutes, 30 minutes, 1 hour, etc.) and n can be any number selected by a user that is possible given the duration of time series training data. In some embodiments, a confidence score for each increment of l may be provided, to designate to users that the confidence in pain intensity in a more imminent timeframe will likely be higher than the confidence in pain intensity in more distant timeframes. Based on the trained third AI, the third AI can predict the pain classification indication and/or the pain intensity at a future time based on the current sensor data. In other embodiments, the third AI model may comprise both a pain classification/intensity model that determines current pain level as well as a prediction model that determines a future pain level curve. In some embodiments the model used to predict future pain intensity may comprise a Recurrent Neural Network or similar deep learning technique, or may include statistical methods such as an ARIMA-based method. For example, an expected pain curve may be initially set for each subject based upon factors such as age, weight, type of operation, etc. The curve could be presented visually to caregivers in a hospital setting, and would continually update as pain assessments are made for the subject at the cadence/periodicity determined per the techniques described above. The expected pain curve, being a form of multivariate time series prediction, could also depict confidence levels for future time internals: For example, the visualization may indicate a 90% likelihood of a 2/10 pain intensity during the next 10 minutes, an 80% likelihood of a 3/10 pain intensity during the subsequent 10 minutes, and so forth.

Using the expected pain curve, a software system can be implemented that would assist caregivers in determining optimal times to provide minimal pain treatment, thereby improving outcomes for neonates. In other words, the processes 200A, 200B, and/or 300 can be relied upon for implementing a system for suggesting timing, dosage, and medication type for pain relief medication treatment, through automatic and real-time pain assessment. In some embodiments, the system can alert caregivers (e.g., by audible alarm, push notifications, messaging platforms, etc.) if the neonate is experiencing or about to experience pain above a given threshold, such that immediate intervention by medication is needed. Additionally, and perhaps more advantageously, the system can alert caregivers of an optimal time to provide a minimal pain intervention, such as a time to provide acetaminophen or ibuprofen, or other non-opiate, to prevent the subject from reaching a pain level that would require a higher degree of intervention. In this sense, the multivariate time-series model can be trained to take into account effect of medication treatment on the expected pain curve, to keep neonates from having an intensity of pain that would necessitate opiates.

In further examples, the process 200A, 200B, and/or 300 can reduce the length of stay in NICU by effectively controlling pain relief medications. In further examples, the process 200A, 200B, and/or 300 can predict pain and avoid a neonate getting into meaningful pain in the first place. Furthermore, the process 200A, 200B, and/or 300 can perform individual pain predictions for different states (sleeping, hungry, wet diaper, etc.) based on different modalities and training data.

In even further examples, this example approach could be used for assessing and predicting pain for other populations of people unable to verbally respond to pain, either due to physical (e.g., stroke, traumatic brain injury), mental (e.g., Down's syndrome), or cognitive (e.g., dementia) disabilities.

In the foregoing specification, implementations of the disclosure have been described with reference to specific example implementations thereof. It will be evident that various modifications may be made thereto without departing from the broader spirit and scope of implementations of the disclosure as set forth in the following claims. The specification and drawings are, accordingly, to be regarded in an illustrative sense rather than a restrictive sense.

What is claimed is:

1. A method for pain assessment comprising:
obtaining a trained first artificial intelligence (AI) model, a trained second AI model, and a trained third AI model;
obtaining data of a plurality of data modalities corresponding to a time period;
generating a latent feature of the data for each modality of the plurality of data modalities based on the trained first AI model and the data for each modality of the plurality of data modalities;
generating a common latent space based on the trained second AI model and the latent feature space of each modality of the plurality of data modalities;
generating a reconstructed latent feature for a missing modality based on the common latent space and the trained second AI model; and
determining a pain indication based on the latent feature for each modality of the plurality of data modalities, the reconstructed latent feature for the missing modality, and the trained third AI model.

2. The method of claim 1, wherein the data of the plurality of data modalities comprises a plurality of intermediate features corresponding to the plurality of data modalities, the plurality of intermediate features produced from a plurality of sub-AI models, the plurality of intermediate features corresponding to the plurality of sub-AI models.

3. The method of claim 2, further comprising:
obtaining sensor data of the plurality of data modalities corresponding to the time period,
wherein the data of the plurality of data modalities is determined based on the sensor data of the plurality of data modalities and the plurality of sub-AI models.

4. The method of claim 3, wherein the plurality of data modalities comprises at least one of: a face modality, a body modality, or an auditory modality.

5. The method of claim 4, wherein the sensor data of the plurality of data modalities comprises at least one of: a plurality of facial images for the face modality, a plurality of body images for the body modality, or audio data for the auditory modality.

6. The method of claim 1, wherein the trained first AI model comprises an autoencoder neural network, and
wherein the latent feature for each modality of the plurality of data modalities comprises an output of an encoder in the autoencoder neural network.

7. The method of claim 1, wherein the trained second AI model comprises a variational autoencoder neural network,
wherein the method further comprises:
generating a probability distribution of the latent feature for each modality of the plurality of data modalities based on an inference model of the variational autoencoder neural network; and
generating a joint-posterior distribution based on the probability distribution for each modality of the plurality of data modalities,
wherein the common latent space is generated based on the joint-posterior distribution.

8. The method of claim 1, wherein the trained third AI model comprises a transformer encoder neural network,
wherein the method further comprises:
stacking the latent feature for each modality of the plurality of data modalities and the reconstructed latent feature of the missing modality;
generating a plurality of attentive features based on the trained third AI model and the stacked latent feature for each modality of the plurality of data modalities with the reconstructed latent feature; and
concatenating the plurality of attentive features for a final feature vector,
wherein the pain indication is determined based on the final feature vector.

9. The method of claim 1, wherein the pain indication comprises at least one of: a pain classification or a level of pain intensity.

10. The method of claim 1, further comprising:
obtaining a vital sign; and
producing a sepsis indication based on the pain indication and the vital sign.

11. A method for pain assessment artificial intelligence (AI) model training comprising:
obtaining training data of a plurality of data modalities corresponding to a time period;
obtaining a ground truth pain indication for the training data;
training a first AI model based on the training data of the plurality of data with unsupervised learning, the first AI model configured to generate a latent feature of the training data for each modality of the plurality of data modalities;
training a second AI model based on the second AI model and the latent feature space of each modality of the plurality of data modalities with unsupervised learning, the second AI model configured to generate a common latent space and a reconstructed latent feature for a missing modality; and
training a third AI model based on the latent feature for each modality of the plurality of data modalities, the reconstructed latent feature, and the ground truth pain indication for the missing modality, the third AI model configured to generate a pain indication.

12. The method of claim 11, wherein the training data of the plurality of data modalities comprises a plurality of intermediate features corresponding to the plurality of data modalities, the plurality of intermediate features produced from a plurality of sub-AI models, the plurality of intermediate features corresponding to the plurality of sub-AI models.

13. The method of claim 12, further comprising:
obtaining sensor data of the plurality of data modalities corresponding to the time period,
wherein the data of the plurality of data modalities is determined based on the sensor data of the plurality of data modalities and the plurality of sub-AI models.

14. The method of claim 13, wherein the time period comprises a plurality of segments, each segment having a same time duration,
wherein the method further comprises:
selecting a same amount of the sensor data for each segment

15. The method of claim 13, wherein the sensor data of the plurality of data modalities comprises at least one of: a plurality of facial images for the face modality, a plurality of body images for the body modality, or audio data for the auditory modality.

16. The method of claim 11, wherein the first AI model comprises an autoencoder neural network,
wherein the latent feature for each modality of the plurality of data modalities comprises an encoder output of an encoder in the autoencoder neural network, and
wherein the first AI model is trained based on a difference between the encoder output of the encoder and a decoder output of a decoder in the autoencoder neural network.

17. The method of claim 11, wherein the second AI model comprises a variational autoencoder neural network, and
wherein the second AI model is trained based on an inference output of an inference model in the variational autoencoder neural network and a generative output of a generative model in the variational autoencoder neural network.

18. The method of claim 17, wherein second AI model is configured to:
generate a probability distribution of the latent feature for each modality of the plurality of data modalities based on an inference model of the variational autoencoder neural network; and
generate a joint-posterior distribution based on the probability distribution for each modality of the plurality of data modalities,
wherein the common latent space is generated based on the joint-posterior distribution.

19. The method of claim 11, wherein the third AI model comprises a transformer encoder neural network,
wherein the method further comprises:
stacking the latent feature for each modality of the plurality of data modalities and the reconstructed latent feature of the missing modality;
generating a plurality of attentive features based on the third AI model and the stacked latent feature for each modality of the plurality of data modalities with the reconstructed latent feature; and
concatenating the plurality of attentive features for a final feature vector,
wherein the pain indication is determined based on the final feature vector, and
wherein the third AI model is trained based on the pain indication and the ground truth pain indication.

20. The method of claim 1, wherein the obtaining of the training data comprises:
obtaining unfiltered data of the plurality of data modalities corresponding to the time period; and
selecting the training data among the unfiltered data by an influence score of each of the training data being equal or higher than a threshold score.
